(19) United States
(12) Patent Application Publication
(10) Pub. No.: US 2023/0274835 A1
(43) Pub. Date: Aug. 31, 2023

(54) SYSTEM AND METHOD FOR IDENTIFYING PRODROMAL PARKINSON'S DISEASE

(71) Applicant: Altoida, Inc., Washington, DC (US)

(72) Inventors: Ioannis Tamanas, Washington, DC (US); Maximilian Bugler, Schechen (DE); Robbert Harms, Nijmegen (NL)

(21) Appl. No.: 18/109,810

(22) Filed: Feb. 14, 2023

Related U.S. Application Data
(60) Provisional application No. 63/309,909, filed on Feb. 14, 2022.

(51) Int. Cl. G16H 50/20; G16B 20/00; G16B 40/20; G16H 10/20

(52) U.S. Cl. CPC G16H 50/20; G16B 20/00; G16B 40/20; G16H 10/20

(57) ABSTRACT

A method of generating a digital biomarker signature for identification of a prodromal condition of a disease includes the steps of: obtaining a plurality of sets of digital biomarkers for preclinical Alzheimer's disease; selecting a subset of the plurality digital biomarkers; applying the subset of digital biomarkers to a group of subjects exhibiting a disease other than Alzheimer's; ranking the biomarkers of the subset on the basis of the commonality of each biomarker in said group of subjects; determining a digital biomarker signature indicative of said disease on the basis of the subset of the plurality of biomarkers and the ranking thereof. The other disease may be Parkinson's disease, and the subject of digital biomarkers may be deemed indicative of prodromal Parkinson's disease. An embodiment may use detection and prediction models for Alzheimer's disease to the identification of prodromal Parkinson's disease using explainable artificial intelligence.

### CLAIM OF PRIORITY UNDER 35 U.S.C. § 119

The present application for patent claims priority to Provisional Application No. 63/309,909 entitled “SYSTEM AND METHOD FOR IDENTIFYING PRODROMAL PARKINSON'S DISEASE” filed Feb. 14, 2022, and assigned to the assignee hereof and hereby expressly incorporated by reference herein.

### TECHNICAL FIELD

The present invention relates generally to a system and method of determining prodromal disease, in some embodiment to the detection of prodromal Parkinson's disease. In some embodiments, the system and method use detection and prediction models for Alzheimer's disease and applies these to the identification of prodromal Parkinson's disease in particular in subjects not suffering from Alzheimer's disease.

### BACKGROUND OF THE INVENTION

Parkinson's disease (PD) is the fastest growing neurodegeneration illness. Parkinson's disease exhibits a pre-diagnostic phase in which it is difficult to identify clinical and laboratory biomarkers for those people in the earliest stages of the disease or those who are at risk.

The prevalence of Parkinson's disease (PD) increases with age and rises from about 1% in individuals aged 60+ to 3.5% in older adults of 85-89 years of age. The complexity of cross-sectional diagnosis is stereotypically exemplified in Parkinson's disease, which happens to be the second most common neurodegenerative disorder after Alzheimer's disease. The pathological hallmark of Parkinson's disease is misfolded alpha-synuclein protein (aSyn) structures, and the gold standard for diagnosis is their identification in post-mortem pathological examinations of the brain. However, given that most idiopathic patients experience years or sometimes decades of unspecific symptomology, attempts are being made in the field of pre-diagnostic Parkinson's disease (Prodromal-PD) to try to discover a panel of clinical and laboratory biomarkers for those at risk. Multiple different strategies are being investigated but with non-optimal success. Prodromal-PD is when individuals do not fulfil diagnostic criteria for Parkinson's disease (that is, bradykinesia and at least one other motor sign) but exhibit signs and symptoms that indicate a higher-than-average risk of developing motor symptoms and a diagnosis of Parkinson's disease in the future. Presently, most imaging markers across a range of modalities and the emerging literature on fluid and peripheral tissue biomarkers is limited in their ability to predict Prodromal Parkinson's disease, pointing to a need to identify robust predictors of change across the entire spectrum from ordinary to symptomatic Parkinson's disease for more realistic primary or secondary preventive trials for Parkinson's disease.

### SUMMARY OF THE PRESENT INVENTION

The present invention seeks to provide an improved system and method of identifying prodromal illness, particularly prodromal Parkinson's disease.

According to an aspect of the present invention, there is provided a method of generating a set of digital biomarkers for identification of a prodromal condition of a disease comprising the steps of:
obtaining a plurality of sets of digital biomarkers for preclinical Alzheimer's disease;
selecting a subset of the plurality digital biomarkers for preclinical Alzheimer's disease;
applying the subset of digital biomarkers for preclinical Alzheimer's disease to a group of subjects exhibiting a disease other than Alzheimer's;
ranking the biomarkers of the subset on the basis of the commonality of each biomarker in said group of subjects;
determining a new set digital biomarkers indicative of said disease on the basis of the subset of the plurality of biomarkers and the ranking thereof.

In some embodiments, the other disease may be Parkinson's disease and the subject of digital biomarkers may be intended to be indicative of prodromal Parkinson's disease.

The method may include the step of determining which Alzheimer's disease biomarkers may also relate to prodromal Parkinson's disease and selecting the subset of biomarkers on the basis of said determined biomarkers.

In some embodiments, the subset of biomarkers may be selected on the basis of biomarkers deemed predictive of both preclinical Alzheimer's disease and the biological mechanism of a-synucleinopathy in Prodromal-PD.

The method may include the step of selecting biomarkers deemed predictive of both preclinical Alzheimer's disease in a group of patients at different stages of Alzheimer's disease.

The method may include the step of testing the preclinical Alzheimer's disease biomarkers on a group of healthy patients and generating at least one digital biomarker signature for the group of healthy patients, the method including the step of determining the subset of biomarkers for prodromal Parkinson's disease on the basis of the digital biomarker signatures of both the group of preclinical Alzheimer's disease patients and the group of healthy patients.

In some embodiments, the selection may be based upon the generation of SHapley Additive exPlanations (SHAP).

In some embodiments, the subset of biomarkers may be selected on the basis of measures of pre-motor symptoms and behavioural/cognitive decline.

The subset of biomarkers may be selected to include:
(i) a first contributing group of digital biomarker features indicative of brain network function and navigation micro-errors;
(ii) a second group of digital biomarker features relating to Augmented Reality global telemetry variance, representative of coarse-scale hand motion micro-movement;
(iii) a third group of digital biomarker features comprising frequency magnitudes during object placement.

In some embodiments, the subset of digital biomarkers may be selected to take into account age and speed of motor function.

The subset of biomarkers may be selected to identify micro-errors and micro-movements detectable by Fourier analysis on accelerometer data that are not visible to the naked eye or to a camera system.

According to another aspect of the present invention, there may be provided an apparatus for generating a set of digital biomarkers for identification of prodromal Parkinson's disease, the apparatus comprising a processing unit configured:
to obtain a plurality of sets of digital biomarkers for preclinical Alzheimer's disease;
to select a subset of the plurality digital biomarkers;
to apply the subset of digital biomarkers to a group of subjects exhibiting a disease other than Alzheimer's;
to rank the biomarkers of the subset on the basis of the commonality of each biomarker in said group of subjects;
to determine a digital biomarker signature indicative of said disease on the basis of the subset of the plurality of biomarkers and the ranking thereof.

In some embodiments, the other disease may be Parkinson's disease and the subject of digital biomarkers is intended to be indicative of prodromal Parkinson's disease.

In some embodiments, the processing unit may be configured to determine which Alzheimer's disease biomarkers may also relate to prodromal Parkinson's disease and to select the subset of biomarkers on the basis of said determined biomarkers.

In some embodiments, the processing unit may be configured to select the subset of biomarkers on the basis of biomarkers deemed predictive of both preclinical Alzheimer's disease and the biological mechanism of a-synucleinopathy in Prodromal-PD.

The processing unit may be preferably configured to select biomarkers deemed predictive of both preclinical Alzheimer's disease in a group of patients at different stages of Alzheimer's disease.

In some embodiments, the processing unit is configured to process preclinical Alzheimer's disease biomarkers from a group of healthy patients and to generate at least one set of digital biomarkers for the group of healthy patients, and to determine the subset of biomarkers for prodromal Parkinson's disease on the basis of the sets of digital biomarkers of both the group of preclinical Alzheimer's disease patients and the group of healthy patients.

In a practical embodiment, the processing unit may be configured to make the selection based upon the generation of SHapley Additive exPlanations (SNAP).

In some embodiments, the processing unit may be configured to select the subset of biomarkers on the basis of measures of pre-motor symptoms and behavioural/cognitive decline.

In some embodiments, the processing unit may be configured to select the subset of biomarkers to include:
(i) a first contributing group of digital biomarker features indicative of brain network function and navigation micro-errors;
(ii) a second group of digital biomarker features relating to Augmented Reality global telemetry variance, representative of coarse-scale hand motion micro-movement;
(iii) a third group of digital biomarker features comprising frequency magnitudes during object placement.

In some embodiments, the processing unit may be configured to select the subset of digital biomarkers to take into account age and speed of motor function.

The processing unit may be configured to select the subset of biomarkers to identify micro-errors and micro-movements detectable by Fourier analysis on accelerometer data that are not visible to the naked eye.

The features and advantages described in the specification are not all-inclusive. In particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter. Other aspects and features of the invention and teachings herein will be apparent to the skilled person from the specific description which follows.

### BRIEF DESCRIPTION OF THE DRAWINGS

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts. Embodiments of the present invention are described below, by way of example only, in which:

FIG. 1 is a flowchart showing the overall dataset structure of a preferred embodiment of biomarker processing method for use in the identification of prodromal Parkinson's disease;

FIG. 2 is a schematic diagram of apparatus configured to implement the method of FIG. 1;

FIG. 3 is a representation of the motoric functioning tasks used in an embodiment of tests that can be performed in the system and method taught herein;

FIG. 4 is an illustration of an augmented reality (AR) task preferably used by the system and method taught herein;

FIG. 5 shows a set of features determined as important by the system and method as an effective Prodromal-PD classifier; and

FIG. 6 shows an example of a digital fingerprint used in an example for the generation of a novel digital biomarker signature for the identification of Prodromal Parkinson's disease.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DESCRIPTION OF THE PREFERRED EMBODIMENTS

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Despite the current research efforts, the inventors believe further progress in this field hinges on a more effective application of digital biomarker and artificial intelligence applications at the pre-diagnostic stages of Parkinson's disease. It is of the highest importance to stratify such pre-diagnostic subjects that might have the most neuroprotective benefit from drugs. However, current initiatives to identify individuals at risk or in the earliest stages of the disease and who might be candidates for future clinical trials are still challenging due to the limited accuracy and explainability of existing pre-diagnostic detection and progression prediction solutions.

A method of generating a digital biomarker signature for identification of a prodromal condition of a disease includes the steps of: obtaining a plurality of sets of digital biomarkers for preclinical Alzheimer's disease; selecting a subset of the plurality digital biomarkers; applying the subset of digital biomarkers to a group of subjects exhibiting a disease other than Alzheimer's; ranking the biomarkers of the subset on the basis of the commonality of each biomarker in said group of subjects; determining a digital biomarker signature indicative of said disease on the basis of the subset of the plurality of biomarkers and the ranking thereof. The other disease may be Parkinson's disease, and the subject of digital biomarkers may be deemed indicative of prodromal Parkinson's disease. An embodiment may use detection and prediction models for Alzheimer's disease to the identification of prodromal Parkinson's disease using explainable artificial intelligence. The disclosed systems and methods demonstrate that it is possible to detect a novel digital biomarker signature from existing datasets using digital biomarker data collected from preclinical Alzheimer's patient data sets.

The present invention in its preferred embodiments provides a novel set of digital biomarkers, of a type equivalent to the Digital Neuro Signature (DNS) used by the Applicant's systems and methods, for identification, in the preferred embodiment, of prodromal Parkinson's disease (Prodromal-PD) based on selected digital biomarkers previously discovered for Preclinical Alzheimer's disease (AD). Results obtained by the applicant have demonstrated the ability to generate a common digital biomarker signature for both Preclinical Alzheimer's disease and Prodromal-PD, containing a ranked selection of features. In the preferred embodiments, this novel digital biomarker signature has been rapidly repurposed out of 793 digital biomarker features, as a selection of the top 20 digital biomarkers that are deemed by the inventors as predictive and able to detect both the biological signature of Preclinical Alzheimer's disease and the biological mechanism of a-synucleinopathy in Prodromal-PD. The novel digital biomarker signature can be used in identifying subjects who may have prodromal Parkinson's disease independently of Alzheimer's disease, that is subjects who do not have or who may not be at risk of developing Alzheimer's disease.

The resulting model can provide physicians with a pool of patients potentially eligible for Parkinson's disease therapy. It demonstrates the importance of digital biomarkers that are predictive, preferably determined on the basis of SHapley Additive exPlanations (SNAP). The inventors believe that similar initiatives could clarify the stage before and around diagnosis, enabling the field to push into unchartered territory at the earliest stages of Parkinson's disease.

Longitudinal measures of pre-motor symptoms and behavioural/cognitive decline are essential for evaluating preclinical markers and monitoring Prodromal-PD progression.

Such longitudinal characterization of non-motor features has been identified by the Movement Disorders Society (MDS) as being valuable for early identification of Parkinson's disease, according to the Research Criteria for Prodromal-PD, which include two types of measurements: the delineation of the relative temporal trajectories of specific quiet motor and non-motor features that can be present before diagnosis, and the fluctuation of those features over time within and across neurocognitive domains.

The utility of such markers in evaluating Prodromal-PD progression depends on early symptoms and signs before Parkinson's disease diagnosis is possible and may vary across different primary care settings. However, intra-individual variability (IIV) across several measurements, called dispersion, is a sensitive marker for detecting change even at prodromal stages of a disease. One digital biomarker tool that utilizes dispersion to provide such measurements is the Altoida Digital Neuro Signatures platform (DNS), a more efficient, accurate, and sensitive assessment of cognitive function than traditional neuropsychological tests, both in cross-sectional and longitudinal evaluations. Previous studies have validated the machine learning model's performance to measure dementia disease progression and detect the biological signature of Prodromal Alzheimer's disease, which predicts conversion from mild cognitive impairment (MCI) to Alzheimer's disease (AD) with 94% prognostic accuracy.

The emergence of large longitudinal primary care cohorts, alongside advances in digital biomarkers and artificial intelligence (AI), has allowed detailed exploration of the full range of early motor and non-motor symptoms that predate Parkinson's disease. In contrast, advanced Prodromal-PD detection and prediction models could become a platform for medical practitioners wishing to diagnose or detect the disease earlier and more accurately.

Despite the enthusiasm that objective motor dysfunction occurs prior to diagnosis of Parkinson's disease and the variety of measuring devices that have been developed, including software applications that aim to harness passive and active digital biomarker sensors for capturing activity, motion, measure gait, bradykinesia, dyskinesia and nocturnal movement, there has remained a lack of effective identification of pre-diagnostic Parkinson's disease.

The aim of the developments leading to this invention has been to determine whether detection and prediction models for Alzheimer's disease could be applied to the identification of prodromal Parkinson's disease using explainable artificial intelligence. A technical hurdle in the development of the preferred embodiments has been to ensure that any detection and prediction model is configured to focus both on improving system performance and AI interpretability, as well as employing natural language explanations to help physicians understand the predictions.

For this purpose, the inventors focused, in an embodiment, on digital biomarker signature patterns between existing databases and ANANEOS data (explained in the next paragraph) data using permutation-based techniques to help understand the actual effect of the predictors (digital biomarker signatures from the existing Alzheimer's disease database) in the target database (preclinical markers that predict prodromal Parkinson's disease progression). This sought to identify digital biomarker signature similarities from previous studies and a dataset collected in the ANANEOS Project.

ANANEOS is an ongoing single-centre, observational, longitudinal cohort (n=500,000) for individuals (50+) with a ClinicalTrials.gov Identifier: NCT04701177. It is an ambitious longitudinal community-based study for healthy aging in Greece. The ANANEOS Project is part of the GR2021 Priority project “Healthy Brains for life” (Age 20-99) and focuses on the decentralized and remote assessment of the symptoms of preclinical stages in Alzheimer's disease and movement disorders with a rationale and a methodology similar to other international initiatives. The participants, recruited initially since March 2021 in Athens, Greece, are home-dwelling volunteers with known biological and psychological biomarkers at the preclinical stages in Alzheimer's disease and movement disorders without relevant psychiatric, neurological, or systemic disorders.

It will be appreciated that other data sets could equally be used. Other examples of similar large-scale national initiatives can be found in Japan with the IROOP registry system for identifying risk factors for dementia, the Sidney (Australia) memory and ageing study, the Framingham heart study in the US, the UK Biobank study of lifestyle and genetic factors incidence in dementia, the European Prevention of Alzheimer's Dementia Longitudinal Cohort Study, the FINGER project in Finland, the INTERCEPTOR Project in Italy and The Vallecas Project in Spain.

Data Collection

In this example, a combination of previously collected clinical and population data was used, from previous studies by the Applicant. The clinical data (n=438) consists of controlled tests of elderly (50+) subjects with known biological and psychological biomarkers (e.g., MCI, amyloid-beta (Ab)+, Ab−, AD). A dataset described as “New validation study” (ClinicalTrials.gov Identifier: NCT02843529) was used for this work, the original purpose of which was to evaluate the performance of the applicant's technology as an adjunctive tool for diagnosing Alzheimer's disease. This data was collected in various major cities in Italy, Greece, Spain, USA, and Ireland.

That dataset was enriched with two more databases:
1) A clinical dataset called RADAR-AD (see Muurling, M., de Boer, C., Kozak, R. et al. Remote monitoring technologies in Alzheimer's disease: design of the RADAR-AD study. Alz Res Therapy 13, 89 (2021). https://doi.org/10.1186/s13195-021-00825-4). RADAR-AD is a multi-centre observational, cross-sectional, cohort study in subjects within the preclinical-to-moderate Alzheimer's disease spectrum as well as healthy controls. The design entails three tiers: (1) a main study, which includes smartphone applications and wearable devices only; (2) a first sub-study, which includes in addition data from fixed sensors at the participant's home; and (3) a second sub-study, which includes in addition data from fixed sensors in an existing smart home environment. Participating clinical sites were selected based on their geographic location, expertise in digital technologies and disease population of interest, and the availability of clinical cohorts with known Alzheimer's disease biomarkers; and
2) A population dataset collected by the applicant and named “healthy basket”. In this example, the healthy basket was a population sample consisting of middle-aged cognitively healthy Japanese subjects (n=130). The inclusion criteria for participation were age 20-50 and self-assessed cognitively healthy (that is, having no known cognitive disorders). All subject information was anonymized and deidentified. Beyond the digital biomarkers collected, no further biomarkers were recorded for this population sample. For both datasets, the subject's sex was self-reported. All subjects (of both groups) performed multiple test sessions.

Finally, the target database was part of the Digitally-enhanced, Decentralized, Multi-omics Observational Cohort (ANANEOS) study.

Reference is now made to Table 1 below.

TABLE 1

| | | | Men | | Women | | Total | P-value |
|---|---|---|---|---|---|---|---|---|
| Population | Clinical data | N (%) | 280 | (42%) | 378 | (58%) | 658 | 1.2e−12 |
| | Population | N (%) | 94 | (72%) | 36 | (28%) | 130 | |
| | data | | | | | | | |
| | ANANEOS | N (%) | 66 | (50%) | 67 | (50%) | 133 | |
| Age | | Mean | 56.9 | (17.4) | 62.7 | (12.8) | 60.1 | 7.5e−06 |
| | | (SD) | | | | | | |
| Status | Healthy | N (%) | 198 | (45%) | 237 | (55%) | 435 | 0.786 |
| | Preclinical | N (%) | 103 | (46%) | 117 | (54%) | 220 | |
| | AD | | | | | | | |
| | MCI ab− | N (%) | 16 | (38%) | 26 | (62%) | 42 | |
| | MCI ab+ | N (%) | 35 | (44%) | 43 | (56%) | 78 | |
| | AD | N (%) | 5 | (38%) | 8 | (62%) | 13 | |
| | Prodromal- | N (%) | 13 | (54%) | 11 | (46%) | 24 | |
| | PD | | | | | | | |
| Number of | | N (%) | 1448 | (52%) | 1359 | (48%) | 2807 | — |
| digital | | | | | | | | |
| biomarker | | | | | | | | |
| signature | | | | | | | | |
| trials (data | | | | | | | | |
| points) | | | | | | | | |
| Number of | | Median | 2 | (4) | 2 | (5) | 2 (3) | 2.8e−05 |
| digital | | (IQR) | | | | | | |
| biomarker | | | | | | | | |
| signature | | | | | | | | |
| trials (data | | | | | | | | |
| points) per | | | | | | | | |
| subject | | | | | | | | |

In Table 1, the P-value is calculated using a two-sided t-test for age, chi2 for status and the Mann-Whitney rank test for the number of data points per subject.

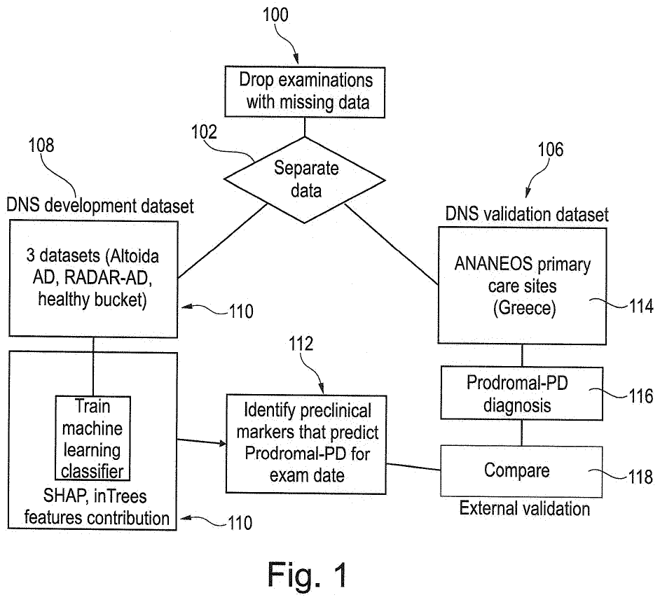

Table 1 describes the data characteristics for the entire sample and stratified by sex, with univariate comparisons. The data in this example consists of 788 subjects combined from three datasets, two clinical datasets and a healthy population dataset. Subjects were distributed over several stages of the Alzheimer's disease clinical continuum, namely healthy, Preclinical AD Ab+, MCI (amyloid-beta negative) Ab−, MCI Ab+, dementia due to Alzheimer's disease and Prodromal-PD as reported by clinical assessment. To counter the imbalance from multiple data points per subject and combining two demographically different datasets, the process stratified all analysis by dataset, sex, and number of data points. This ensured that there would be the same number of data points from each sex and from each study (clinical and population). The flowchart showing the overall dataset structure and the preliminary study purpose is shown in FIG. 1.

With reference to FIG. 1, at step 100 the process initially discards data sets having missing data. At step 102, the remaining data sets are separated into a digital biomarker signature development dataset 104 and a digital biomarker signature validation dataset 106.

The digital biomarker signature development dataset comprises three data sets 108, all relating to Alzheimer's disease. In this example the three datasets comprise: (i) the Applicant's Alzheimer's disease dataset, (ii) a RADAR Alzheimer's data set and (iii) a data set from a healthy cohort of subjects. At step 110 these three datasets are processed as disclosed herein, specifically to identify biomarkers that are considered potentially appropriate for the identification of prodromal Parkinson's disease. The processing is as explained below. The process results in the selection of a subset of preclinical Alzheimer's disease biomarkers that are considered able to predict prodromal Parkinson's disease, as shown at step 112.

The digital biomarker signature validation dataset, in this embodiment, comprises an ANANEOS dataset (at 114), as described above. This dataset is intended to identify subjects exhibiting clinically identifiable prodromal Parkinson's disease and is used as a comparator for the identified biomarkers. Only those subjects who are known to have Parkinson's disease are selected for the digital biomarker signature validation dataset.

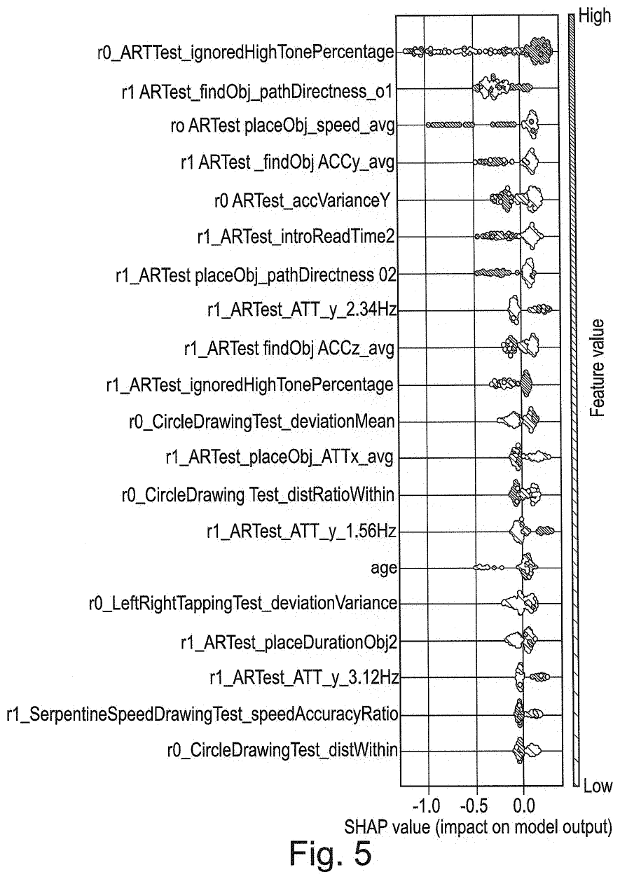

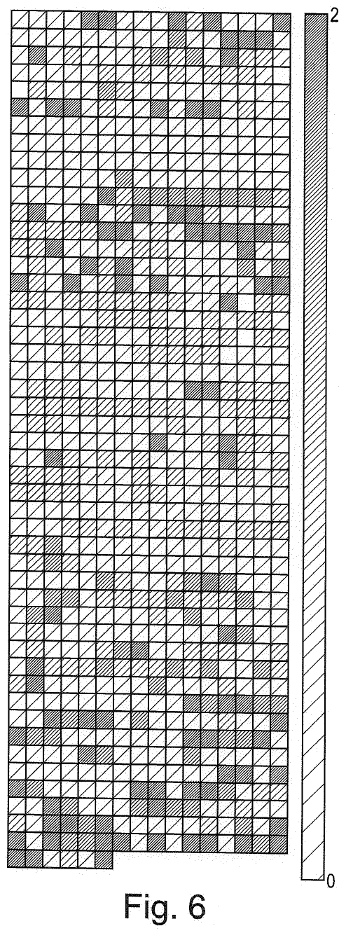

At step 118, the process applies the subset of digital biomarkers determined at steps 110 and 112 to the subjects diagnosed with clinically observable prodromal Parkinson's disease (step 116) and determines which biomarkers of the subset can be observed in those subjects. The outcome of this comparison at step 118 is used to rank the biomarkers in the subset in order of importance. Those biomarkers which are most commonly observed in the group of subjects are given a high ranking, while those that are less common in the group of subjects are given a lower ranking. FIG. 5, described below, shows the ranked results of 20 digital biomarkers obtained by the process disclosed herein in a preferred subset determined at steps 110 and 112.

It will be appreciated that if the comparison step 118 fails to identify reasonable correspondence between the subset of biomarkers and the subjects from step 116, the assumptions made in the subset selection in steps 110 and 112 can be revised in order to seek to identify a different subset of biomarkers.

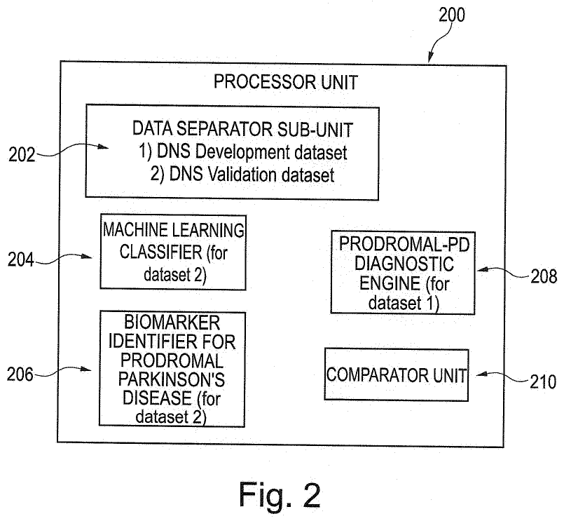

FIG. 2 shows an example of apparatus configured to implement the method of FIG. 1. The apparatus comprises components configured to store and/or process data in accordance with the described method. In summary, the apparatus comprises: a processing unit 200 that includes a data separator sub-unit 202 for separating the datasets into Development and Validation datasets (steps 104 and 106 of FIG. 1), a machine learning classifier 204, a biomarker identifier 206, a prodromal-PD diagnostic engine 208 and a comparator unit 210, all configured to perform the taught process steps. These components will typically be electronic and/or computer units of a form which will be evident to the skilled person.

Digital Biomarker Signatures

For this step, data from Altoida's application which collects digital biomarkers for neurocognitive function measurement and predictive diagnosis of Alzheimer's disease was repurposed. Altoida's application collects digital biomarker data for detecting early-onset Alzheimer's disease. Altoida's application is well-known in the art and aspects are described in the applicant's co-pending patent applications, such as: US-2021/196174, EP-2446341, U.S. Ser. No. 63/211,960, U.S. Ser. No. 63/211,953 and U.S. Ser. No. 63/277,456. While holding a tablet or smartphone device, the subject is asked to perform a series of motor functioning tasks and two Augmented Reality (AR) tasks. In the motor functioning tasks, the subject is required to draw shapes and tap on the (touch)screen using the finger of their dominant hand (see FIG. 2 for an illustration of the motor functioning tasks used in this example). In one of the Augmented Reality tasks, the subject is asked to place three virtual objects in a small space (approximately 3 m×3 m or 2 m×4 m) and afterwards find them again.

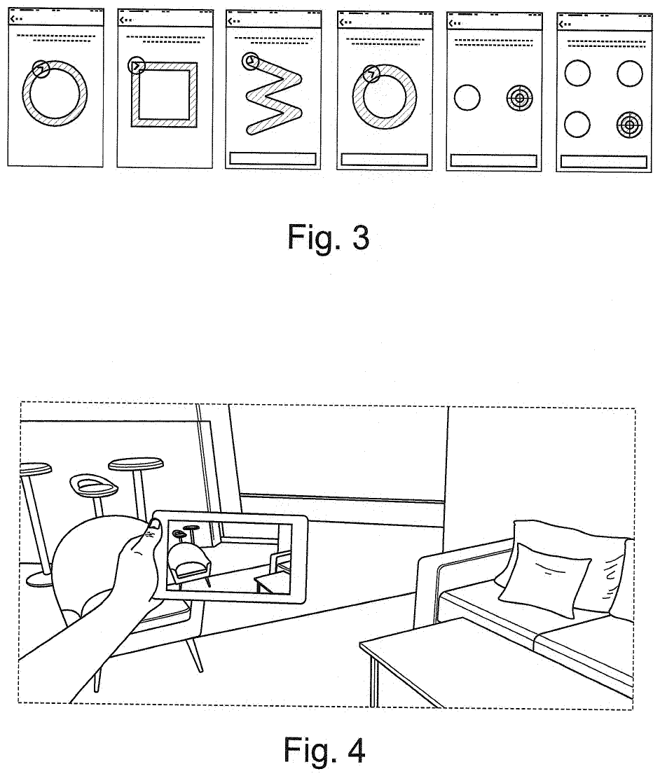

In FIG. 3, the tasks are preferably executed one after another. In this example, using the index finger of their dominant hand, from left to right, the subject's task is 1) to draw a circle, 2) to draw a square, 3) to draw a rotated W shape within 7 seconds, 4) to draw as many circles as possible within 7 seconds, 5) to tap the highlighted buttons (left, right, left, right, etc.) 6) to tap the highlighted button as fast as possible, the buttons being highlighted at random. It will be appreciated that this is just one example of a task-based test for the purposes of the invention disclosed herein.

The Augmented Reality task is performed by navigating around the space with the tablet or smartphone in both hands (see FIG. 4). In this example, during the Augmented Reality test, the subject is asked to place and find three virtual objects in a room. To do so, the subject is required to walk around the room holding a tablet or smartphone device in front of him/her. While doing so, the camera of the device records the environment and displays it back to the user on the screen, augmented with virtual objects (in this illustration, a teddy bear). The user needs to place the objects on flat surfaces and later recall their position by walking back to that location.

During these tasks, the handheld device collects telemetry and touch data from the built-in sensors, enabling profiling of hand micro-movements, screen touch pressures, walking speed, navigation trajectory, cognitive processing speed, and additional proprietary inputs.

A single test session using Altoida's application consisted of two batches of motor tasks and two Augmented Reality tasks. Once a subject has completed all tasks, the recorded digital biomarker data from the onboard electronics sensors is bundled and securely and anonymously uploaded to a server for further processing. Once provided with the data from multiple subjects, machine learning can be used to detect patterns. In previous work, machine learning was either used to classify subjects as healthy or at risk of Alzheimer's disease. Digital biomarker signatures from a previous development dataset for Alzheimer's disease were examined to see if they demonstrated preclinical markers that predict Prodromal-PD progression, expressed by the capacity of results to inform a novel Prodromal-PD digital biomarker signature. This is explained in further detail below.

Machine Learning

In this example, 793 digital biomarker features were extracted from the onboard electronics sensors relating to various cognitive, functional, and physiological characteristics of each subject. These features included, for example, response times, eye-to-hand coordination precision, fluctuations in the telemetry (accelerometer and gyroscope) data, Fourier analysis of the telemetry data, step detection, and additional proprietary data. Based on the digital biomarker feature data from a selection of healthy subjects, a digital biomarker signature match classifier was trained to distinguish prodromal-Parkinson's disease individuals from any other group. In this example, the XGBoost algorithm was used with digital biomarker signature preclinical markers that predict Prodromal-PD as the target variable for the classification. [see Chen, T., & Guestrin, C. (2016). XGBoost: A Scalable Tree Boosting System. In Proceedings of the 22nd ACM SIGKDD International Conference on Knowledge Discovery and Data Mining (pp. 785-794). New York, N.Y., USA: ACM.]

Performance Evaluation

A stratified 5-fold grouped cross-validation was applied to estimate the generalization performance of the digital biomarker signature Prodromal-PD classifier. Data points were grouped by subject to ensure that multiple data points of a single subject were all in the same fold (either training or testing), in order to seek to prevent learning bias. For the Prodromal-PD classifier, accuracy was measured and precision averaged over the five cross-validation testing folds. To assess the classifier's performance on different age groups, nine additional classifiers (10 in total) were trained, each using different random subsets of the data.

Model Explainability

The Shapley Additive exPlanations (SHAP) method was used to understand better the predictions made by the digital biomarker signature Prodromal-PD classifier [see Lundberg, S. M. and Lee, S.-I. (2017). A Unified Approach to Interpreting Model Predictions. In: Guyon, I.; Luxburg, U. V.; Bengio, S.; Wallach, H.; Fergus, R.; Vishwanathan, S. & Garnett, R. (Ed.), Advances in Neural Information Processing Systems 30, Curran Associates, Inc]. The SHAP method allocates to each feature of a classifier a game-theoretical value representing the contribution of that feature towards the classification targets. The sign of the SHAP values indicates the direction of the contribution, while the magnitude of the SHAP value indicates the importance. For the classifier adopted in this example, negative SHAP values contribute to classifying as non-Prodromal-PD, positive numbers towards Prodromal-PD. SHAP values have an additive property meaning they can be summed together to provide the feature contribution of a group of features.

Results

These were intended to generate a common digital biomarker signature for Preclinical Alzheimer's disease and Prodromal-Parkinson's disease and features contribution.

The test of this example was intended to investigate whether detection and prediction models for Alzheimer's disease can be rapidly applied to Prodromal Parkinson's disease using explainable artificial intelligence. The analysis of the Prodromal Parkinson's disease classifier revealed at least 20 common features that are the same for both Preclinical Alzheimer's disease and Prodromal Parkinson's disease. Using this as a basis, a novel digital biomarker signature is generated which can be used in non-Alzheimer subjects to seek to identify prodromal Parkinson's disease in those subjects. After computing a SHAP value for each digital biomarker signature containing more than 793 features from the development dataset that was used, it was possible to reach this conclusion. The identified 20 common features were compared with a novel Prodromal Parkinson's disease classifier, which detected a digital biomarker signature in the ANANEOS validation dataset. It was then investigated which of the 793 features contained the most relevant preclinical markers that could predict Prodromal-Parkinson's disease. FIG. 4 shows a grouping of those features that were ranked as having the highest overall contribution in the classifier.

In this example, the subset of digital biomarkers, being 20 in total in this embodiment, could be classified as biomarkers relating to micro-movements that are not visible to the naked eye or observable by a camera, biomarkers relating to visible movement, subject detectable sound frequency and subject age.

FIG. 4 displays an embodiment of biomarker subset obtainable by the method and system disclosed herein and that could be said to represent a Prodromal Parkinson's disease classifier. FIG. 4 shows: (i) the top twenty feature groups according to the SHAP method, in which each bar represents the summed SHAP value of the features in that feature group; and (ii) a feature value SHAP distribution plot for the top five contributing features. Subject specific SHAP values were computed for each datapoint in the classifier training data. For each feature, the method then plotted for each datapoint a dot with the feature value of that datapoint, with the dot colour coded by the relative feature value. The position of each dot on the SHAP value x-axis represents the magnitude and the direction of the contribution of that specific feature value of that specific datapoint towards classifying as female (−1) or male (+1). Acronyms in the plots are Augmented Reality (AR), Fast Fourier Transform (FFT), SHapley Additive exPlanations (SHAP), Accelerometer (ACC), variance (var), first part of a single test (1st) or second part of a single test (2nd). In FIG. 5, the red areas represent prevalence of the biomarker in prodromal Parkinson's disease patients, while the blue areas represent prevalence of the biomarker in non-prodromal Parkinson's disease subjects.

The subset of digital biomarkers in FIG. 5 are as follows:
ignoredHighTonePercentage: represents the percentage of high pitch sound queues a subject failed to detect or react to;
pathDirectness: represents how close to a straight line the subject moves between objects;
placeObj_speed: represents the time taken for a subject to place an object at a given location;
findObj_ACCy_avg: represents the average measures acceleration in the y-axis while finding an object;
accVariance_y: represents the average variance in the y-axis throughout the entire AR test;
introReadtime: represents the time taken for a subject to read the introductory screens of the application;
CircleDrawingTest_DeviationMean: represents the average deviation from the optimal path in a circle drawing test;
CircleDrawingTest_distRatioWith: represents the ratio of a subject's movement spent within an optimal path area in the circle drawing test;
SerpentineSpeedDrawingTest_speedAccuractRatio: represents the ratio of speed vs accuracy in a Serpentine Speed Drawing Test;
r0_ and r1_ represent first and second rounds of testing, while o1, o2, o3 are objects 1, 2, 3

The twenty biomarkers are listed in order of importance or priority. The top biomarker is that most frequently found in the validation dataset, while the bottom biomarker being that least frequently found, with all the other biomarkers listed in order of prevalence in the validation dataset. This prioritisation of the subset is considered to be particularly useful in the subsequent application of the subset to other subjects for which it is desired to carry out a prodromal Parkinson's disease assessment.

The primary contributing group of features was named the ignore high tone percentage and the Augmented Reality object placement directness. This group consisted of an interference index (non-motor feature) and a set of frequency magnitudes obtained while the participant moved around trying to find a virtual object in the Augmented Reality test (motor feature). These features could therefore be interpreted as a brain network function and navigation micro-errors.

The second most important group of digital biomarker features is the AR global telemetry variance. The global telemetry variance is the variance in the accelerometer and gyroscope signal over the entire duration of the AR task. It could be interpreted as coarse-scale hand motion micro-movement (motor feature).

The third and fourth most important feature sets were frequency magnitudes during object placement, belonging to the top group placing virtual objects in the Augmented Reality test, collected using a Fast Fourier Transform (FFT) on the measured accelerometer and gyroscope signal over 1.28 seconds before placing (motor feature).

The remaining elements of the novel digital biomarker Prodromal-PD signature take into account age and group together “Motor test drawing features” to consider the speed and accuracy of the subject while drawing various patterns with the index finger (motor feature). The “Circle drawing test” measures how long the user spent within the limits of the circle while performing the motor tests.

### Practical Example

Referring now to FIG. 5, this shows an example of a Digital Neuro Fingerprint (DNF) of the Applicant's, representative of the aggregated results of a series of digital biomarker signatures from, in this example, 100 patients, obtained from a set of around 800 biomarkers. This digital fingerprint is used in one embodiment in the generation of a novel digital biomarker signature for prodromal Parkinson's disease.

Assignment of weights for the approximately 800 digital biomarkers obtained, based on the colour frequency of appearance at the individual digital biomarker signatures, which complete the prototypical digital fingerprint. For example, if there is a digital fingerprint that consists of 100 digital biomarker signatures and the digital biomarker no1 is appearing as green at 99 of those 100 digital biomarker signatures, then this digital biomarker (no1) is weighted as #1 for the colour green. The same process is followed for all digital fingerprint matrix colours: green, orange, red etc. Once the weights per colour have been assigned for the top 20 digital biomarkers per colour, a decision tree is used for the creation of just one list that consists of different coloured biomarkers that interact with each other the most. For example, if the #1 for Green interacts the most with #2 for orange and #10 for red in 90 of the 100 digital biomarker signatures, this group is assigned at #1 in the new list (if no other interaction is more frequent). The process continues until there is a combined list of 20 digital biomarkers of different colours, based on their interactions.

During this process, novel insights can be created about biomarker interactions that were not obvious to experts before. Such interactions could create a novel meta-biomarker.

In the event that some frequencies per biomarker are matching, for example two or three of the digital biomarkers that are coloured green appear at 99 of those 100 digital biomarker signature sand the implementation cannot decide which one of the two or three is to be ranked #1, the system assigns the weight there as #1.1, #1.2, #1.3 etc. At the next level in the process the combined list biomarker interactions is processed to resolve any ambiguities from the previous level. In the statistically unlikely event that even at the next level of biomarkers interaction an ambiguity exists, for example a specific interaction of two or more from the X colour interaction groups appears in 90 of the 100 digital biomarker signatures above, then an expert's opinion is used in order to resolve the ambiguity and rank the weight of those two or more interactions for the combined list above.

Treatments

The early detection of Parkinson's Disease, particularly Prodromal Parkinson's Disease, can enable the early administration of treatments. While there is currently no known cure for Parkinson's Disease, treatments exist which can reduce the speed of onset of the disease and alleviate the symptoms. It is believed that the earlier a treatment can be administered, the better will be the outcome for the patient.

Treatments may include: supportive therapies, such as physiotherapy, medication, and surgery (occasionally).

Supportive therapies are intended to make living with Parkinson's disease easier and to help the patient deal with their symptoms on a day-to-day basis. Supportive therapies include: physiotherapy, which can relieve muscle stiffness and joint pain through movement and exercise. Occupational therapy can be used to identify areas of difficulty in a patient's everyday life, such as getting dressed/undressed and carrying out practical daily chores and routines. Other supportive therapies include speech and language therapy, usually caused by the patient experiencing dysphagia (swallowing difficulties). Speech and language therapy can help improve these problems by providing speaking and swallowing exercises or by providing assistive technology.

Medicaments, or drugs, can be used to improve the main symptoms of Parkinson's disease, such as shaking (tremors) and movement problems. There are currently the main types of medication that are commonly used, namely: levodopa, dopamine agonists and monoamine oxidase-B inhibitors. Levodopa is absorbed by the nerve cells in the patient's brain and turned into dopamine, which is used to transmit messages between the parts of the brain and nerves that control movement. Increasing the levels of dopamine usually improves movement problems. Levodopa can often be combined with other medication, such as benserazide or carbidopa, which stop the levodopa being broken down in the bloodstream before it has a chance to get to the brain. They can also alleviate side effects of levodopa, which may include: nausea, tiredness, dizziness. Alternatively, or additionally to levodopa, dopamine agonists may be prescribed, which act as a substitute for dopamine in the brain and have a similar but milder effect compared with levodopa. They can often be given less frequently than levodopa. Dopamine agonists can be taken as a tablet and are available also as a skin patch (rotigotine). Alternatively, or additionally to Levodopa and/or dopamine agnostics, the patient can be prescribed monoamine oxidase-B (MAO-B) inhibitors, such as selegiline and rasagiline, which are another alternative to levodopa for treating early Parkinson's disease. Monoamine oxidase-B (MAO-B) inhibitors block the effects of an enzyme or brain substance that breaks down dopamine (monoamine oxidase-B), increasing dopamine levels. Apomorphine, is a dopamine agonist called that can be injected subcutaneously by a single injection, when required or a continuous infusion using a small pump carried around on the patient's belt, clothing or in a bag. For severe Parkinson's, a type of levodopa called co-careldopa may be pumped continuously into the patient's gut through a tube inserted through the patient's abdominal wall.

In some cases, the patient may benefit from surgery, specifically deep brain stimulation. This involves surgically implanting a pulse generator similar to a heart pacemaker into the patient's chest wall. It is connected to 1 or 2 fine wires placed under the skin and is inserted precisely into specific areas in the patient's brain. A tiny electric current is produced by the pulse generator, which runs through the wire and stimulates the part of the patient's brain affected by Parkinson's disease.

More specifically, drugs used in the treatment of Parkinson's disease may be grouped by type (class). These are levodopa, dopamine agonists, MAO-B Inhibitors, COMT inhibitors, amantadine and anticholinergics. Co-careldopa (Sinemet) is a common levodopa medication, for example.

Dopamine is a chemical messenger made in the brain. The symptoms of Parkinson's Disease appear when dopamine levels become too low, due to many of the cells in the patient's brain that produce dopamine dying or having died. Taking dopamine as a drug doesn't work because it cannot cross the blood brain barrier. To get around this, doctors use other medication that can act in a similar way. Most drug treatments work by increasing the amount of dopamine in the brain and/or acting as a substitute for dopamine by stimulating the parts of the brain where dopamine works, by blocking the action of other factors (enzymes) that break down dopamine.

Drugs that may be prescribed include: Carbidopa-levodopa (Rytary, Sinemet, Duopa, others). Levodopa is the most effective Parkinson's disease medication, as it is a natural chemical that passes into the patient's brain and is converted to dopamine. Levodopa is typically used with carbidopa (Lodosyn), which protects levodopa from early conversion to dopamine outside the patient's brain. This can ameliorate side effects such as nausea.

Inhaled carbidopa-levodopa may also be used, such as Inbrija™, alternatively an infusion may be used, such as Duopa™, which combines carbidopa and levodopa. It can be administered through a feeding tube that delivers the medication in a gel form directly to the small intestine.

Dopamine agonists include pramipexole (Mirapex ER), and rotigotine (Neupro, given as a patch). Apomorphine (Apokyn) is a short-acting injectable dopamine agonist used for quick relief.

MAO B inhibitors may include selegiline (Zelapar), rasagiline (Azilect) and safinamide (Xadago). They help prevent the breakdown of brain dopamine by inhibiting the brain enzyme monoamine oxidase B (MAO B). This enzyme metabolizes brain dopamine. Selegiline given with levodopa may help prevent wearing-off.

Catechol O-methyltransferase (COMT) inhibitors include Entacapone (Comtan) and opicapone (such as Ongentys), which are the primary medications in this class. This medication can prolong the effect of levodopa therapy by blocking an enzyme that breaks down dopamine.

Tolcapone (such as Tasmar) is another COMT inhibitor that is rarely prescribed due to a risk of serious liver damage and liver failure. Several anticholinergic medications are available, including for example benztropine (Cogentin) or trihexyphenidyl. Amantadine (such as Gocovri) may be prescribed alone to provide short-term relief of symptoms of mild, early-stage Parkinson's disease. It may also be given with carbidopa-levodopa therapy during the later stages of Parkinson's disease to control involuntary movements (dyskinesia) induced by carbidopa-levodopa. Adenosine receptor antagonists (A2A receptor antagonist) target areas in the brain that regulate the response to dopamine and allow more dopamine to be released. Istradefylline (Nourianz) is a known A2A antagonist drug. Nuplazid (Pimavanserin) can be used to treat hallucinations and delusions that can occur with Parkinson's disease.

Deep brain stimulation involves the implantation of electrodes into a specific part of the brain. The electrodes are connected to a generator implanted in the patient's chest near the collarbone, to send electrical pulses to the patient's brain and may reduce Parkinson's disease symptoms. Deep brain stimulation is often offered to people with advanced Parkinson's disease who have unstable medication (levodopa) responses. It can stabilize medication fluctuations, reduce or halt involuntary movements (dyskinesia), reduce tremor, reduce rigidity, and improve movements.

Advanced treatments include MRI-guided focused ultrasound (MRgFUS), which is a minimally invasive treatment that has helped some people with Parkinson's disease manage tremors. Ultrasound is guided by an MRI machine to the area in the brain where the tremors start. The ultrasound waves are at a very high temperature and burn areas that are contributing to the tremors.

### CONCLUSION

The method demonstrates that it is possible to detect a novel digital biomarker signature from existing datasets using digital biomarker data collected from preclinical Alzheimer's patient data sets. The identified intrinsic similarities between Preclinical Alzheimer's disease markers and preclinical markers that can predict Prodromal Parkinson's disease seem to be capturing quiet motor and non-motor features dependent on age. In the pre-diagnostic Parkinson's disease population, the primary differentiating features have been identified as micro-errors and micro-movements detectable by Fourier analysis on accelerometer data, although they are non-visible to the naked eye. Such early identifiable results from this method can provide physicians with some insights into driving factors of prediction models from multiple points of view including visualization and feature importance based, for example, on SHapley Additive exPlanations (SNAP).

The words used in this specification to describe the instant embodiments are to be understood not only in the sense of their commonly defined meanings, but to include by special definition in this specification: structure, material or acts beyond the scope of the commonly defined meanings. Thus, if an element can be understood in the context of this specification as including more than one meaning, then its use must be understood as being generic to all possible meanings supported by the specification and by the word or words describing the element.

The definitions of the words or drawing elements described above are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

In the foregoing description and in the figures, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used above, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B”, when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, processes, operations, values, and the like.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

1. A method of generating a digital biomarker signature for identification of a prodromal condition of a disease comprising the steps of:
obtaining a plurality of sets of digital biomarkers for preclinical Alzheimer's disease;
selecting a subset of the plurality of sets of biomarkers;
applying the subset of the plurality of sets digital biomarkers to a group of subjects exhibiting a disease other than Alzheimer's;
ranking the digital biomarkers of the subset on a basis of a commonality of each biomarker in said group of subjects; and
determining a digital biomarker signature indicative of said disease on the basis of the subset of the plurality of sets of digital of biomarkers and the ranking thereof.

2. A method according to claim 1, wherein an other disease is Parkinson's disease and the subject of digital biomarkers is deemed indicative of prodromal Parkinson's disease.

3. A method according to claim 1, including the step of determining which Alzheimer's disease biomarkers may also relate to prodromal Parkinson's disease and selecting the subset of biomarkers on the basis of said determined biomarkers.

4. A method according to claim 3, wherein the subset of biomarkers is selected on a basis of biomarkers deemed predictive of both preclinical Alzheimer's disease and a biological mechanism of a-synucleinopathy in Prodromal-PD.

5. A method according to claim 3, including the step of selecting biomarkers deemed predictive of both preclinical Alzheimer's disease in a group of patients at different stages of Alzheimer's disease.

6. A method according to claim 3, including the step of testing the preclinical Alzheimer's disease biomarkers on a group of healthy patients and generating at least one digital biomarker signature for the group of healthy patients, the method including the step of determining the subset of biomarkers for prodromal Parkinson's disease on the basis of the digital biomarker signature of both the group of preclinical Alzheimer's disease patients and the group of healthy patients.

7. A method according to claim 3, wherein the selection is based upon a generation of SHapley Additive exPlanations (SHAP).

8. A method according to claim 3, wherein the subset of biomarkers is selected on a basis of measures of pre-motor symptoms and behavioural/cognitive decline.

9. A method according to claim 3, wherein the subset of biomarkers is selected to include:
(i) a first contributing group of digital biomarker features indicative of brain network function and navigation micro-errors;
(ii) a second group of digital biomarker features relating to Augmented Reality global telemetry variance, representative of coarse-scale hand motion micro-movement; and
(iii) a third group of digital biomarker features comprising frequency magnitudes during object placement.

10. A method according to claim 3, wherein the subset of the plurality of sets digital biomarkers is selected to take into account age and speed of motor function.

11. A method according to claim 1, wherein the subset of biomarkers is selected to identify micro-errors and micro-movements detectable by Fourier analysis on accelerometer data that are not visible to a naked eye.

12. Apparatus for generating a digital biomarker signature for identification of prodromal Parkinson's disease, the apparatus comprising a processing unit configured:
to obtain a plurality of sets of digital biomarkers for preclinical Alzheimer's disease;
to select a subset of the plurality of sets of digital biomarkers;
to apply the subset of digital biomarkers to a group of subjects exhibiting a disease other than Alzheimer's;
to rank the digital biomarkers of the subset on a basis of a commonality of each biomarker in said group of subjects; and
to determine a digital biomarker signature indicative of said disease on the basis of the subset of the plurality of sets of digital biomarkers and the ranking thereof.

13. Apparatus according to claim 12, wherein an other disease is Parkinson's disease and the subject of digital biomarkers is deemed to be indicative of prodromal Parkinson's disease.

14. Apparatus according to claim 13, wherein the processing unit is configured to determine which Alzheimer's disease biomarkers may also relate to prodromal Parkinson's disease and to select the subset of biomarkers on the basis of said determined biomarkers.

15. Apparatus according to claim 14, wherein the processing unit is configured to select the subset of biomarkers on a basis of biomarkers deemed predictive of both preclinical Alzheimer's disease and a biological mechanism of a-synucleinopathy in Prodromal-PD.

16. Apparatus according to claim 14, wherein the processing unit is configured to select biomarkers deemed predictive of both preclinical Alzheimer's disease in a group of patients at different stages of Alzheimer's disease.

17. Apparatus according to claim 14, wherein the processing unit is configured to process preclinical Alzheimer's disease biomarkers from a group of healthy patients and to generate at least one digital biomarker signature for the group of healthy patients, and to determine the subset of biomarkers for prodromal Parkinson's disease on the basis of the digital biomarker signature of both the group of preclinical Alzheimer's disease patients and the group of healthy patients.

18. Apparatus according to claim 14, wherein the processing unit is configured to make the selection based upon a generation of SHapley Additive exPlanations (SNAP).

19. Apparatus according to claim 14, wherein the processing unit is configured to select the subset of biomarkers on a basis of measures of pre-motor symptoms and behavioural/cognitive decline.

20. Apparatus according to claim 14, wherein the processing unit is configured to select the subset of biomarkers to include:
(i) a first contributing group of digital biomarker features indicative of brain network function and navigation micro-errors;
(ii) a second group of digital biomarker features relating to Augmented Reality global telemetry variance, representative of coarse-scale hand motion micro-movement; and
(iii) a third group of digital biomarker features comprising frequency magnitudes during object placement.

21. Apparatus according to claim 14, wherein the processing unit is configured to select the subset of digital biomarkers to take into account age and speed of motor function.

22. Apparatus according to claim 13, wherein the processing unit is configured to select the subset of biomarkers to identify micro-errors and micro-movements detectable by Fourier analysis on accelerometer data that are not visible to a naked eye.

23. A method of treating a subject determined to have a degenerative disease, the method comprising:
(i) obtaining results of a method of generating a digital biomarker signature for identification of a prodromal condition of a disease, said method comprising the steps of:
obtaining a plurality of sets of digital biomarkers for preclinical Alzheimer's disease;
selecting a subset of the of sets of plurality digital biomarkers;
applying the subset of digital biomarkers to a group of subjects exhibiting a disease other than Alzheimer's;
ranking the biomarkers of the subset on a basis of a commonality of each biomarker in said group of subjects;
determining a digital biomarker signature indicative of said disease on the basis of the subset of the plurality of biomarkers and the ranking thereof; and
administering a treatment for reducing progression of the disease to the subject determined to have mild cognitive impairment due to the disease.

24. A method according to claim 23, for treatment of a subject found to have at least one of: Prodromal Parkinson's Disease and Parkinson's Disease in a subject.

25. A method according to claim 23, including administration of a treatment for reducing progression of Parkinson's Disease to a subject determined to have mild cognitive impairment due to Parkinson's Disease.

26. A method according to claim 25, including the administration of at least one of: levodopa, dopamine agonists and monoamine oxidase-B inhibitors, benserazide, carbidopa.

27. Apparatus according to claim 12, for use in a diagnosis of cognitive impairment due to at least one of: Prodromal Parkinson's Disease and Parkinson's Disease in a subject.

28. Apparatus according to claim 12, for use in an administration of a treatment for reducing progression of Parkinson's Disease to a subject determined to have mild cognitive impairment due to Parkinson's Disease.
